(19) United States
(12) Patent Application Publication
(10) Pub. No.: US 2004/0144387 A1
(43) Pub. Date: Jul. 29, 2004

(54) Double-balloon endobronchial catheter for one lung isolation anesthesia and surgery

(76) Inventor: David Amar, Hillcrest, NY (US)

Correspondence Address: KLAUBER & JACKSON, 411 HACKENSACK AVENUE, HACKENSACK, NJ, 07601

(21) Appl. No.: 10/353,120

(22) Filed: Jan. 28, 2003

(51) Int. Cl. A61M 16/00

(52) U.S. Cl. 128/207.14; 606/192

(57) ABSTRACT

A double balloon endobronchial catheter for one lung anesthesia and surgery. A catheter tube has distal and proximal ends and a first inflatable balloon at the distal end thereof and a second inflatable balloon spaced a predetermined distance proximally from the first balloon, with each balloon being independently inflatable. The catheter is introduced into the patient through an endotracheal tube to position the first balloon within the bronchus intermedius and the second inflatable balloon at or just proximal of the upper lobe of the bronchus. Both balloons, when inflated, occlude flow at two differing, spaced apart, locations within the bronchus to isolate that lung for surgery. A method is disclosed that inflates the first balloon to stabilize the location of the catheter and then inflates second balloon. The predetermined distance between the two balloons insures that the second balloon is correctly located when inflated.

### BACKGROUND OF THE INVENTION

The present invention relates to catheters, and more particularly, to a double-balloon endobronchial catheter that is used to isolate one lung of a patient during anesthesia and surgery.

There are many operations that are carried out or performed today on a patient that require one lung of that patient to be isolated, that is, one of the patient's lungs must be kept relatively immobilized during the operation so that the surgery can be performed. Typical of such operations include thoracoscopic lung surgery, minor or major lung resection, thoracic aorta repair or reconstruction, esophageal surgery and anterior thoracic spine surgery.

The need to isolate such lungs in order to operate stems from the very nature of a surgical operation performed under general anesthesia. In such operations, an anesthesia apparatus actually breathes for the patient by means of an anesthesia ventilator that acts in conjunction with an anesthesia machine. The anesthesia ventilator provides a timed, intermittent flow of gas to the patient to force that gas into the patient to expand the lungs, while the gas is expelled from the lungs as the lungs deflate. The anesthesia machine adds the anesthetic, in the form of a vapor, to the gas so that the anesthetic laden gas is provided to the patient to carry out the induction and maintenance of anesthesia in the patient during the operation. Thus under normal operation, both lungs are continually expanded and contracted during the inhalation and exhalation of the anesthetic laden gases in accordance with the normal cycling of the anesthesia ventilator.

Obviously, with the aforelisted operations where it is desirable prevent the normal motion of one of the lungs, it becomes necessary to isolate that lung for the entire operation so that it is not subject to the normal expansion and contraction that is caused by the anesthesia ventilator. Therefore, the lung to be operated on is normally isolated by occluding the bronchus of that lung such that the anesthesia machine breathes the anesthetic laden gas into and out of the other lung without affecting the lung subject to the operation. Thus the patient can be anesthetized and the operation can be carried out on the isolated lung. One of the difficulties, however, is to fully and effectively occlude the mainstem bronchus leading into the lung that is to be subject to the surgery.

One of the more common techniques currently used to provide the necessary isolation for one lung is through the use of a double lumen endotracheal tube. With the use of a double lumen endotracheal tube, however, there are certain drawbacks to its use.

In particular, there can be a considerable drawback in its insertion in the event of an unanticipated difficult airway visualization or the presence of a paralyzed vocal cord. Further, the anesthesiologist may choose to abandon the use of a double lumen endotracheal tube in the event some anatomical problem is encountered in its placement, such as tracheal narrowing, which could preclude the proper positioning of the double lumen endotracheal tube. In addition, with many procedures, there is a difficult situation where the risk of gastric aspiration is high and a double lumen endotracheal tube needs to be replaced by a standard single lumen tube after the conclusion of the operation.

An example of bronchial occlusion by means of a catheter is shown in Arndt, U.S. Pat. No. 5,904,648 where an endobronchial blocker catheter is utilized and is introduced into the patient through a standard endotracheal tube by means of a soft wire-guided loop. The Arndt catheter, however, includes only one balloon and, when inserted in the right main stem bronchus, for example, it would result in a less secure or stable a placement in comparison to the present invention where one catheter with two balloons is used where one, the distal of the two balloons, is advanced further distally into the patient at the level of the right bronchus intermedius of the patient with the second, or proximal balloon, located at or just proximal to the orifice of the right upper lobe in the right main stem bronchus. In addition, of course, once the soft flexible wire-guided loop in Arndt is removed to allow a channel for suction, it cannot be reintroduced into the catheter and thus would preclude repositioning of the Arndt catheter if it becomes dislodged during surgery for whatever reason. Whereas if the catheter is placed by means of a more rigid stylet as proposed in this invention, the stylet can be shaped by the operator for the initial placement of the catheter and later reintroduced to reposition the catheter if it becomes dislodged from the desired location. Also, in Inoue et al, U.S. Pat. No. 4,453,545, an endobronchial blocker is disclosed, however, there is, again, only one blocker within the main bronchus.

A method and catheter for achieving one-lung isolation by means of occluding the bronchus at two differing locations through the use of a double endobronchial catheter is shown and described in co-pending U.S. patent application Ser. No. 09/878,788, filed Jun. 11, 2001 and entitled DOUBLE ENDOBRONCHIAL CATHETER FOR ONE LUNG ISOLATION ANESTHESIA AND SURGERY, owned by the assignee of the present application and the disclosure of that patent application is incorporated herein by reference. In the aforementioned patent application, a pair of blocking catheters are used in connection with a bifurcated outer sheath and each of the blocking catheters is individually manipulated to be positioned at predetermined locations or sites within the main stem bronchus of the patient.

While the dual blocking catheter approach of the aforesaid application produces a viable product and is effective to achieve the goal of one lung isolation, the use of two individual blocking catheters raises issues with respect to manufacturability and requires the individual maneuvering of each of the blocking catheters by means of a stylet or the like generally guided by the use of a fiber optic bronchoscope and thus requires some careful maneuvering to locate both of the blocking catheters in the desired location where they are effective to carry out the lung isolation.

Accordingly, it would be desirable to have a catheter that could be readily manufactured and which is also relatively easy to be introduced through a tracheal tube that has been intubated into a patient such that the actual positioning of the operative components of the catheter can be easily located at the desired site to occlude the main stem bronchus of the patient at two spaced apart locations without the need to carry out the individual positioning of separate blocking catheters.

### SUMMARY OF THE INVENTION

Thus, in accordance with the present invention, there is provided a double balloon endobronchial catheter that is relatively easy to use with a patient and yet which provides effective isolation of a single lung of the patient so that surgery can be performed on that lung without interference caused by movement of that lung during the normal general anesthesia involving the ventilation of the lung.

With the present invention, there is a catheter tube that is generally a flexible plastic tube having a distal end and a proximal end, and, as is conventional, the distal end is adapted to be introduced into the patient while the proximal end is accessible external of the patient. In use, therefore, the present double balloon endobronchial catheter is adapted to be introduced into the patient through the normal endotracheal tube to be positioned such that the distal end is located within the main stem bronchus of the patient that communicates with the lung to be isolated.

At or proximate to the distal end of the catheter tube, there is located a first inflatable balloon while a second inflatable balloon is affixed to the catheter tube at a predetermined distance proximal to the first inflatable balloon, that is, the first and second balloons are spaced apart along the catheter tube a predetermined distance to carry out a purpose that will be later explained. Both of the balloons are separately and individually inflatable and thus, there can be separate inflating lumens that are provided in the catheter tube, and preferably, and conventionally, there are two lumen that are molded into the wall of the catheter tube to carry out the inflating process with both inflating lumens extending from the interior of the balloons to suitable fittings external of the patient for connection to an inflation device, such as a syringe.

The overall catheter is flexible and can be manipulated by the user proximal to the patient by means such as a stylet that can be passed through a main passageway formed in the catheter tube and removed after the catheter has been properly positioned. The main passageway can also be used for the suctioning of fluids from the lung of the patient where needed by means of a vacuum source or system that can be connect to the proximal end of the catheter tube. Alternatively, a positive pressure can be applied to the main passageway to partially or fully inflate the lung when necessary.

In accordance with the present invention, there is provided a double balloon endobronchial catheter wherein there is a first and a second inflatable balloon where each balloon can be positioned readily at specific desired, spaced apart, locations within the main stem bronchus of a patient and where each balloon is inflatable to create a blockage at a particular desired site within the main stem bronchus. Thus, the spacing or distance between the first and second inflatable balloons is a critical element with respect to the present endobronchial catheter since, in operation, the first inflatable balloon is intended to be located within the main stem bronchus of the patient at the bronchus intermedius to block the main stem bronchus at that site. The second inflatable balloon, that is located the predetermined distance proximal to the first inflatable balloon, is intended to be located within the main stem bronchus at or just proximal to the upper lobe orifice.

Thus, as can be seen, the distance between the balloons is a critical element of the present invention and which is measured by the mid portion of the balloons, generally where sealing contact is made with the internal surface of the main stem bronchus. In the preferred embodiment, the distance between the first and second inflatable balloons is between about 1 to 2 centimeters and the more preferred distance is about 1.5 centimeters.

Accordingly, by the use of the catheter tube having two inflatable balloons located in the main stem bronchus of the patient, the main stem bronchus of the patient can be occluded in two different and spaced apart locations.

In the process of introducing the double balloon endobronchial catheter of the present invention into a patient, a unique method can be employed due to the presence of the two inflatable balloons that both are adapted to be located in the main stem bronchus and independently inflatable. Thus, after a standard endotracheal tube has been placed into a patient in normal fashion, the distal end of the double balloon endobronchial catheter can be introduced through a standard endotracheal tube, using a fiber optic bronchoscope for visual guidance and the double balloon endobronchial catheter progressed into the main stem bronchus until the first inflatable balloon, that is, the balloon that is at the distal end of the catheter, is properly located in the bronchus intermedius. The first inflatable balloon (distal) can then be inflated such that the double balloon endobronchial catheter is basically fixed, or stabilized, in that position within the patient. The second inflatable balloon (proximal) can then be inflated and the physician will be assured that the second balloon will be automatically located in the more difficult of the two balloon positions, that is, at or just proximal to the upper lobe orifice due to the predetermined distance between the first inflatable balloon and the second inflatable balloon. Accordingly, by stabilizing the catheter by inflation of the first inflatable balloon, the subsequent inflation of the second inflatable balloon will be automatically located in the desired site within the main stem bronchus at or just proximal to the upper lobe orifice.

As will be seen, the inflation of both the first and second inflatable balloons will effectively isolate that lung so that one lung surgery can be accomplished since the main stem bronchus is effectively occluded at two, spaced apart, locations.

Other features of the overall double balloon endobronchial catheter will become more apparent in light of the following detailed description of a preferred embodiment thereof and as illustrated in the accompanying drawings.

### BRIEF DESCRIPTION OF THE DRAWINGS

FIG. 1 is a schematic view of a double balloon endobronchial catheter constructed in accordance with the present invention shown in its operative position intubated into a patient;

FIG. 2 is a cross-sectional view of the double balloon endobronchial catheter of FIG. 2.

### DETAILED DESCRIPTION OF THE INVENTION

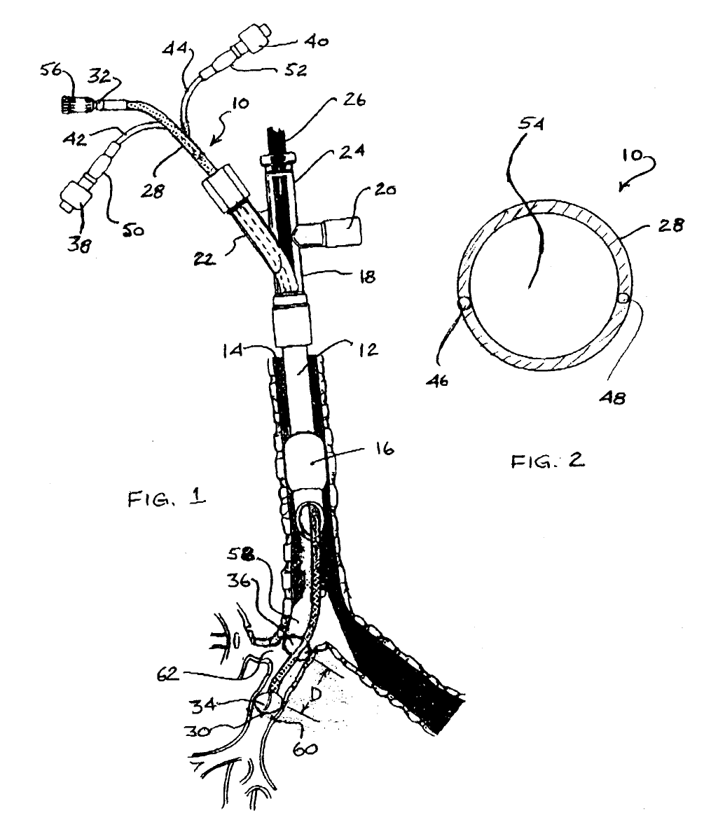

Referring now to FIG. 1, there is shown a schematic view of a double balloon endobronchial catheter 10 in its operative position within a patient. As can be seen, an endotracheal tube 12 is positioned within the trachea 14 and has a tracheal cuff 16 that is inflated to seal the endotracheal tube 12 within the trachea 14 of the patient. Accordingly, ventilation can be carried out through proximal openings or connections to the anesthesia ventilator.

In the embodiment shown, the double balloon endobronchial catheter 10 is adapted to be intubated into the patient by sliding the double balloon endobronchial catheter 10 through an airway adapter 18 having a port 20 to be connected to a breathing circuit, a side passageway 22 and a central passageway 24 through which a fiber optic bronchoscope 26 can be positioned in order for the physician to visually perceive the various passageways of the patient in order to properly position the present invention in a manner that will later be explained.

In FIG. 1, taken along with FIG. 2, the endobronchial catheter 10 includes a catheter tube 28 having distal end 30 and a proximal end 32. At or proximate to the distal end 30 of the catheter tube 28 there is affixed a first inflatable balloon 34. A second inflatable balloon 36 is also affixed to the catheter tube 28 but displaced proximally a predetermined distance D away from the first inflatable balloon 34.

The first and second inflatable balloons 34, 36 are preferably low pressure, soft balloons which are inflated and deflated by means of a conventional system including fittings 38, 40 into which a syringe can be fitted to force air into or take air from the first and second inflatable balloons 34, 36 through tubing 42, 44 that communicates with the first and second inflatable balloons 34, 36 by lumens 46, 48 extending through the wall of the catheter tube 28 as shown in FIG. 2. The lumens 46, 48 may be extruded into the walls of catheter tube 28 itself. Pilot balloons 50, 52 may also be included to provide an indication to the user that the first and second inflatable balloons 34, 36 are or are not inflated. In view of the relatively small passageways involved with the present invention, the first and second inflatable balloons 34, 36 can be effectively inflated with about 2-5 cc. of air.

As also can be seen, there is a main passageway 54 that extends between the proximal end 32 and the distal end 30 of the catheter tube 28 and the main passageway 54 can be used to withdraw fluids from the lung of a patient, and also to accommodate a stylet to assist in the proper positioning of the distal end 30 of the catheter tube 28 within the patient. At the proximal end 32 of the catheter tube 28, therefore, there may be a suction port 56 for connection to a source of vacuum to carry out the removal of fluids.

As mentioned, the main passageway 54 can also be used to introduce a stylet therethrough to provide assistance in guiding the distal end 30 of the catheter tube 28 to its desired site within the patient and, after that desired location has been attained, the stylet can be removed whereupon the main passageway 54 is thereafter usable for the suction of fluids or, of course for the application of positive pressure as desired in order to inflate the lung fully or partially as required during the course of the surgical operation.

With the use of a stylet, not shown, the stylet can be controlled and adjusted at the proximal end 32 of the catheter tube 28 by the physician in order to manipulate the distal end 30 of the double balloon endobronchial catheter 10 such that the distal end 30 can be positioned to locate the first inflatable balloon 34 in the position as shown in FIG. 1, that is, in the main stem bronchus 58 at the bronchus intermedius 60.

With the first inflatable balloon positioned within the bronchus intermedius 60, the predetermined distance D between the first and second inflatable balloons 34, 36 automatically locates the second inflatable balloon 36 within the main stem bronchus 58 at or just proximal to the upper lobe orifice 62. At that position, the first and second inflatable balloons 34, 36 can be inflated such that the upper lobe orifice 62 and the bronchus intermedius 60 can both be occluded together or individually.

With the aforegoing description of the double balloon endobronchial catheter 10 an explanation of the use of the present invention can now be set forth. In carrying out the method steps of the present invention, the endotracheal tube 12 is intubated into the trachea of the patient using conventional procedures.

Once the endotracheal tube 12 is properly positioned the tracheal cuff 16 can be inflated to secure and seal the endotracheal tube 12 within the trachea 14 of the patient. The double balloon endobronchial catheter 10 is thereupon introduced through the side passageway 22 by means such as a stylet interfitted within the main passageway 54 of the catheter tube 28 and which can be aided visually by the use of a fiber optic bronchoscope 26. In the preferred method, when the first inflatable balloon 34 has been properly located with the bronchus intermedius 60, i.e. the desired location for that balloon, the first inflatable balloon 34 is inflated, thereby securing and stabilizing the double balloon endobronchial catheter 10 in that position.

The then stabilized double balloon endobronchial catheter 10 is therefore in the desired position and the second inflatable balloon 36 can be inflated. The location of the second inflatable balloon 36 is, as explained, in a fixed, predetermined distance proximal from the first inflatable balloon 34 on the catheter tube 28 so that the second inflatable balloon 36 is automatically located at the desired site, i.e. at or just proximal to the upper lobe orifice 62 so as to block the upper lobe orifice 62.

In locating the first and second inflatable balloons 34, 36, the more difficult site is the site or location for the second inflatable balloon 36, that is, it is more difficult to position the second inflatable balloon 36 at the correct location at or just proximal to the upper lobe orifice 62 and therefore by initially inflating the first inflatable balloon 34 at the bronchus intermedius, the double balloon endobronchial catheter 10 is stabilized and therefore when the second inflatable balloon 36 is inflated, the predetermined distance D assures that the second inflatable balloon 36 will be properly positioned so that both the bronchus intermedius 60 and the upper lobe orifice 62 are blocked or occluded by the inflation of both the first and second inflatable balloons 34, 36.

The difficulty arises in the lesser definition of the site for the second inflatable balloon, the is within the main stem bronchus 58 at or just proximal to the upper lobe orifice 62. While there are, of course, differences between patients, the bronchus intermedius 60 in generally can be about 2.0 to 4.0 centimeters in length and therefore the first inflatable balloon 34 can fairly readily be located in the desired position therein.

The location of the second inflatable balloon 36, however, at or just proximal to the upper lobe orifice 62 has a range or critical distance of about 1.8 centimeters plus or minus about 0.8 cm and thus may vary from 1.0 to 2.6 centimeters in length and therefore is a more difficult target to position the second inflatable balloon 36. By initially locating and inflating the first inflatable balloon 34, the second inflatable balloon 36 is automatically positioned within the desired range of the main stem bronchus at or just proximal to the upper lobe orifice 62.

Accordingly, by now inflating the first and second inflatable balloons 34, 36 the upper lobe orifice 62 and the bronchus intermedius 60 can both selectively and individually be occluded so that there is an isolation of one of the lungs of the patient and induction and general anesthesia can be carried out on the patient with the use only of the other lung. At the same time the physician also has available the main passageway 54 of the double balloon endobronchial catheter 10 in the event the physician desires to apply suction to the lung or even to add air to partially inflate the lung so that the lung is at the proper state of inflation or deflation to best carry out the operation.

While the present invention has been set forth in terms of a specific embodiment or embodiments, it will be understood that the double balloon endobronchial catheter and the method of isolating one lung of a patient may be modified or altered by those skilled in the art to other configurations or methods. Accordingly, the invention is to be broadly construed and limited only by the scope and spirit of the claims appended hereto.

I claim:

1. A double balloon endobronchial catheter for isolating a lung of a patient by blocking the main stem bronchus of a patient at two spaced apart locations, said catheter comprising:
a catheter tube having a proximal end and a distal end,
a first inflatable balloon affixed to the catheter tube and located at or proximate to the distal end thereof,
a second inflatable balloon affixed to the catheter tube and located a predetermined distance proximal to the first inflatable balloon,
the predetermined distance separating the first and second inflatable balloons being determined to allow the catheter tube to be located within the main stem bronchus of a patient with the first inflatable balloon at a first predetermined location within the main stem bronchus and the second inflatable balloon at a second predetermined location within the main stem bronchus of a patient,
wherein each of said first and second inflatable balloons are separately and independently inflatable to block, respectively, the main stem bronchus of a patient at two spaced apart locations.

2. The double balloon endobronchial catheter as defined in claim 1 wherein the predetermined distance separating the first and second inflatable balloons is determined by the distance between the first predetermined location at the bronchus intermedius and the second predetermined location at or just proximal to the upper lobe orifice of a patient.

3. The double balloon endobronchial catheter as defined in claim 2 wherein the predetermined distance between the first and second balloons is between about 1 and 2 centimeters.

4. The double balloon endobronchial catheter as defined in claim 3 wherein the predetermined distance is about 1.5 cm.

5. The double balloon endobronchial catheter as defined in claim 2 wherein the double balloon endobronchial catheter includes a main passageway extending between the proximal and distal ends of the catheter tube.

6. The double balloon endobronchial catheter as defined in claim 5 wherein the double balloon catheter includes a stylet that is adapted to be located within the main passageway to aid in the manipulation of the distal end of the double balloon catheter.

7. The double balloon endobronchial catheter as defined in claim 1 wherein the catheter tube includes air passages for inflating said first and second balloons.

8. The double balloon endobronchial catheter as defined in claim 7 wherein the stylet is removable.

9. A method of isolating one lung of a patient preparatory to a surgical operation on that isolated lung, said method comprising the steps of:
providing a catheter comprising a catheter tube having a distal end and a proximal end, the catheter tube having first and second inflatable balloons affixed to the catheter tube, with the first inflatable balloon being located at or proximate to the distal end and the second inflatable balloon being located a predetermined distance proximal to the first balloon,
introducing the catheter tube into a patient to position the first inflatable balloon at a first predetermined location within the main stem bronchus of the patient and the second inflatable balloon at a second predetermined location within the main stem bronchus of the patient,
inflating the first and second balloons to block the main stem bronchus of the patient at two separate locations.

10. The method of isolating one lung of a patient as defined in claim 9 wherein said step of introducing the catheter tube comprises introducing the catheter tube to position the first inflatable balloon at the bronchus intermedius of the patient and the second inflatable balloon at or just proximal to the main upper lobe orifice of the patient.

11. The method of isolating one lung of a patient as defined in claim 10 wherein said step of inflating the first and second balloons comprises inflating sequentially the first inflatable balloon to stabilize the catheter tube within the patient and then inflating the second inflatable balloon.

12. The method of isolating one lung of a patient as defined in claim 10 wherein the step of providing a catheter tube comprises providing a catheter tube having a main passageway therethrough and the step of introducing the catheter tube into a patient comprises providing an endotracheal tube, intubating the endotracheal tube into the trachea of the patient and introducing the distal end of the catheter tube through the endotracheal tube.

13. The method of isolating one lung of a patient as defined in claim 10 wherein said step of providing a catheter comprising a catheter tube having a main passageway extending between the proximal and distal ends.

14. A method of isolating one lung of a patient as defined in claim 13 wherein the step of introducing the catheter tube into a patient comprises providing a stylet and inserting the stylet into the main passageway to guide the distal end of the catheter tube into the patient.
